(19) United States
(12) Patent Application Publication
(10) Pub. No.: US 2004/0265243 A1
(43) Pub. Date: Dec. 30, 2004

(54) Cosmetic cleaning foams

(75) Inventors: Harald Albrecht, Hamburg (DE); Stephan Ruppert, Hamburg (DE); Petra Koch, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/812,470

(22) Filed: Mar. 29, 2004

Related U.S. Application Data
(63) Continuation of application No. PCT/EP02/10895, filed on Sep. 27, 2002.

(30) Foreign Application Priority Data
Sep. 29, 2001 (DE) 101 48 392.9

(51) Int. Cl. A61K 7/075; A61K 7/08

(52) U.S. Cl. 424/47; 424/70.22

(57) ABSTRACT

The present invention is a cosmetic cleaning product, comprising a foamable, aqueous cleaning preparation comprising one or more nonionic surfactants and one or more anionic surfactants, and a pumpfoamer comprising a storage container and a pump mechanism in the form of a closure and having a riser tube for foaming the cleaning preparation present in the storage container. The present invention also includes a method of cleansing the skin or hair by applying the cleansing product to the skin or hair through the use of the pumpfoamer.

### CROSS-REFERENCE TO RELATED APPLICATIONS

This is a continuation application of PCT/EP02/10895, filed Sep. 27, 2002, which is incorporated herein by reference in its entirety, and also claims the benefit of German Priority Application No. 101 48 392.9, filed Sep. 29, 2001.

### FIELD OF THE INVENTION

The present invention relates to a cosmetic cleaning agent which is a combination of a filling agent, an application agent and a liquid cleaning preparation and which is converted into a stable foam with the aid of an applicator.

### BACKGROUND OF THE INVENTION

Foams are structures comprising gas-filled, spherical or polyhedral cells which are bounded by liquid, semi-liquid, highly viscous or solid cell walls. The cell walls, connected via so-called node points, form a cohesive framework. Stretched between the cell walls are the foam lamellae (closed-cell foam). If the foam lamellae are destroyed or if they flow back into the cell walls at the end of the foam formation, an open-cell foam is obtained. Furthermore, foams are thermodynamically unstable since surface energy can be gained by decreasing the size of the surface. The stability and hence the existence of a foam is therefore dependent on the extent to which it is possible to prevent its self-destruction.

Cosmetic foams are as a rule dispersed systems of liquids and gases, the liquid representing the dispersant and the gas the dispersed substance. Foams of low-viscosity liquids are temporarily stabilized by surface-active substances (surfactants, foam stabilizers). Owing to their large internal surface area, such surfactant foams have high adsorptivity, which is utilized, for example, in cleaning and washing processes. Accordingly, cosmetic foams are used in particular in the areas of cleaning, for example as shaving foam, or of hair care.

For the production of foam, gas is blown into suitable liquids, or the foam formation is achieved by vigorous beating, shaking, spraying or stirring of the liquid in the relevant gas atmosphere, provided that the liquids contain suitable surfactants or other surface-active substances (so-called foam formers) which, in addition to interfacial activity, also have certain film formation capability. Compared with other cosmetic preparations, cosmetic foams have the advantage that they permit a fine distribution of active substances on the skin. However, cosmetic foams are as a rule achievable only by using particular surfactants, which moreover are often not well tolerated by the skin.

A further disadvantage of the prior art is that such foams are not very stable and therefore usually collapse within about 24 hours. A requirement of cosmetic preparations is, however, that they possess as far as possible stability for years. This problem is generally taken into account by virtue of the fact that the consumer produces the actual foam himself only when required for use, with the aid of a suitable spray system, for which purpose it is possible to use, for example, spray cans in which a liquefied compressed gas serves as a propellant. When the pressure valve is opened, the propellant/liquid mixture escapes through a fine nozzle, and the propellant evaporates and leaves behind the foam.

The preparation of cleaning foams for personal hygiene with the aid of a pumpfoamer (U.S. Pat. No. 6,053,364) from Airspray is known. However, only solutions with a combination of amphoteric and anionic surfactants (WO99/39689) or combinations of nonionic and amphoteric surfactants (U.S. Pat. No. 5,635,469) have been used to date as surfactant solutions. The first version explicitly excludes the use of water-insoluble components, while the second one contains cationic or nonionic polymers as “skin conditioners”.

The use of a combination of anionic and amphoteric surfactants leads to a very cosmetic foam, but also to a strong intrinsic odor which is unpleasant for the user and can be suppressed only by high perfume concentrations. Furthermore, these surfactant solutions have a pale yellow color, which is very unacceptable to many users.

The advantage of foam preparation using a pumpfoamer (U.S. Pat. No. 6,053,364)—compared with the conventional foam preparation by rubbing with the hands—lies in the low concentration of surfactants which is required for obtaining a creamy, fine-pored foam. Consequently, the cleaning foams thus obtained are particularly suitable for cleaning sensitive skin. However, pumpfoamers to date have the disadvantage of contaminating the content with germs since, during the actuation, the pumping process, small amounts of water splashes present in the region of the pump head can be sucked into the pump system.

The foam formation is brought about by mechanical pump systems—as described in WO 00/78629 A1—in which the liquid surfactant preparation is passed at high velocity through a sieve-like fabric or similar structure for fine-pored air enrichment, in order to achieve the desired foam formation. The change in the design of known pumpfoamers can reduce the penetration (aspiration) of water splashes and hence the problem of contamination with germs to such an extent that there is virtually no need to add disinfectant/preservatives/bactericides to the surfactant preparation, which in turn is a decisive advantage in terms of skin tolerance. The protection from water splashes on the foam pump system is brought about by protecting the aeration orifices or channels by a shield-like or roof-like collar, so that splashed water is transported passed the aeration orifices. Furthermore, the collar is formed in such a way that it reliably prevents penetration of water splashes into the inner construction or removes said splashes from the sensitive inner construction, so that contact of the microbiologically contaminated water splashes with the surfactant preparation in any form is ruled out.

The pumpfoamers from Airspray International B.V. are preferred, particularly preferably the pump foamer protected from water splashes (Airspray International B.V., Type WR3).

### SUMMARY OF THE INVENTION

It was not foreseeable for a person skilled in the art that a cosmetic cleaning product comprising

a foamable, aqueous cleaning preparation containing one or more nonionic surfactants and one or more anionic surfactants, and

a pumpfoamer containing a storage container and a pump mechanism in the form of a closure and having a riser tube for foaming the surfactant solution present in the storage container

would remedy the odor and color problems of the prior art, since this combination of surfactants gives an outstanding foam and has virtually no intrinsic odor and is colorless.

### DETAILED DESCRIPTION OF THE PREFERRED EMBODIMENTS

The perfuming of these preparations is readily possible with very low perfume concentrations or, for specific applications, is completely unnecessary. Particularly noteworthy is the synergistic action of the combination of anionic and nonionic surfactants, which, in the combinations according to the invention, leads to a surprisingly and unexpectedly high foam quality—which is intended to denote the amount of foam, the foam stability, and the foam porosity.

Coloring in the colors pleasant for the user is likewise possible by using the conventional dyes in very low concentrations.

The admixing of active substances and auxiliaries is possible within certain concentration ranges without loss of foam quality. Consequently, the combination of cleaning effect and skin care in one product is possible, and a very pleasant skin sensation is achieved after cleaning.

In particular, low concentrations of water-insoluble emollients are in the context of the invention.

Preferably used nonionic surfactant(s) are

1. alcohols,

2. alkanolamides, such as cocamides MEA/DEA/MIPA,

3. amine oxides, such as cocamidopropylamine oxide,

4. esters which form by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glyceryl esters, ethoxylated/propoxylated cholesterols, ethoxylated/propoxylated triglyceride esters, ethoxylated propoxylated lanolin, ethoxylated/propoxylated polysiloxanes, propoxylated POE ethers and alkylpolyglycosides, such as laurylglycoside, decylglycoside and cocoglycoside,

6. sucrose esters, sucrose ethers,

7. polyglyceryl esters, diglyceryl esters, monoglyceryl esters, and

8. methylglucose esters, esters of hydroxy acids.

Preferably, the alkylpolyglycosides, in particular laurylglycoside, decylglycoside and cocoglycoside, have proven advantageous.

The anionic surfactant(s) are, according to the invention, selected from the groups consisting of acylamino acids and salts thereof, carboxylic acids and derivatives thereof, phosphoric esters and salts thereof, sulfonic acids and salts thereof and sulfuric esters.

The compounds mentioned in each case are preferably used as anionic surfactants.

I. acylamino acids (and salts thereof) such as

1. acyl glutamates, for example sodium acylglutamate, di-TEA-palmitoyl aspartate and sodium/caprylic capric glutamate, sodium cocoylglutamate,

2. acyl peptides, for example palmitoyl-hydrolyzed milk protein, sodium cocoyl-hydrolyzed soybean protein and sodium/potassium cocoyl-hydrolyzed collagen,

3. sarcosinates, for example myristoyl sarcosine, TEA-lauroyl sarcosinate, sodium lauroyl sarcosinate and sodium cocoyl sarcosinate,

4. taurates, for example sodium lauroyl taurate and sodium methylcocoyl tau rate,

5. acyl lactylates, lauroyl lactylate, caproyl lactylate, and

6. alaninates;

II. carboxylic acids and derivatives, such as

1. carboxylic acids, for example lauric acid, aluminum stearate, magnesium alkanolate and zinc undecylenate,

2. ester-carboxylic acid, for example calcium stearoyl lactylate, laureth-6 citrate and sodium PEG-4 lauramidocarboxylate, and

3. ether carboxylic acids, for example sodium laureth 13-carboxylate and sodium PEG-6-cocamide carboxylate;

III. phosphoric esters and salts, such as, for example, DEA-oleth-10-phosphate and dilaureth-4 phosphate;

IV. sulfonic acids and salts, such as

1. acyl isethionates, e.g. sodium/ammonium cocoyl isethionate,

2. alkylarylsulfonates,

3. alkylsulfonates, for example sodium cocosmonoglyceride sulfate, sodium C₁₂₋₁₄ olefin-sulfonate, sodium laurylsulfoacetate and magnesium PEG-3 cocamidosulfate, and

4. sulfosuccinates, for example dioctyl sodium sulfosuccinate, disodium laurethsulfosuccinate, disodium laurylsulfosuccinate and disodium undecyleneamido-MEA-sulfosuccinate; and

V. sulfuric esters, such as

1. alkyl ether sulfate, for example sodium, ammonium, magnesium, MIPA and TIPA laureth sulfate, sodium myrethsulfate and sodium C₁₂₋₁₃-parethsulfate, and

2. alkylsulfate, for example sodium, ammonium and TEA laurylsulfate.

Preferably, the acyl glutamates, in particular sodium cocoylglutamate, have proven advantageous.

Surfactant concentrations which are in the range from 2 to 15% by weight, in particular in the range from 4 to 9% by weight (always based on the total weight of the formulation), are particularly advantageous.

The ratio of anionic to nonionic surfactant should be equal to a to b—with a and b being a rational number between 2 and 5. A ratio of anionic to nonionic surfactant of 5 to 3 or of 3.5 to 4, in particular of 2.5 to 3, has proven advantageous.

Oils may be used as skin-care emollients. A possibly desirable oil component of the cosmetic or dermatological cleaning preparations in accordance with the present invention is advantageously selected from the group consisting of the esters of saturated and unsaturated, branched and straight-chain alkanecarboxylic acids having a chain length of 3 to 30 C atoms, saturated and unsaturated, branched and straight-chain alcohols having a chain length of 3 to 30 C atoms, and esters of aromatic carboxylic acids and saturated and unsaturated, branched and straight-chain alcohols having a chain length of 3 to 30 C atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and erucyl erucate as well as synthetic, semisynthetic and natural mixtures of such esters, e.g. jojoba oil

Furthermore, the oil component can advantageously be selected from the group consisting of branched and straight-chain hydrocarbons and hydrocarbon waxes, silicone oils, dialkyl ethers, saturated or unsaturated, branched or straight-chain alcohols and fatty acid triglycerides, namely the triglyceryl esters of saturated and unsaturated, branched and straight-chain alkanecarboxylic acids having a chain length of 8 to 24, in particular 12 to 18, C atoms. The fatty acid triglycerides can, for example, advantageously be selected from the group consisting of the synthetic, semisynthetic and natural oils, e.g. olive oil, sunflower oil, soybean oil, peanut oil, rapeseed oil, almond oil, palm oil, coconut oil, palm kernel oil and the like.

Mixtures of such oil and wax components can also be advantageously used in the context of the present invention. It may also be advantageous to use waxes, for example cetyl palmitate, as the sole lipid component of the oil phase.

The oil component is advantageously selected from the group consisting of 2-ethylhexyl isostearate, octyldodecanol, isotridecyl isononanoate, isoeicosane, 2-ethylhexyl cocoate, C12-15-alkylbenzoate, caprylic/capric acid triglyceride and dicaprylyl ether. Mixtures of C12-15-alkylbenzoate and 2-ethylhexyl isostearate, mixtures of C12-15-alkyl benzoate and isotridecyl isononanoate and mixtures of C12-15-alkyl benzoate, 2-ethylhexyl isostearate and isotridecyl isononanoate are particularly advantageous.

Among the hydrocarbons, liquid paraffin, squalane and squalene can advantageously be used in the context of the present invention.

Advantageously, the oil component can furthermore contain cyclic or linear silicone oils or consist completely of such oils, but it is preferable to use an additional content of other oil phase components apart from the silicone oil or the silicone oils.

Cyclomethicone (octamethyldicyclotetrasiloxane) is advantageously employed as a silicone oil to be used according to the invention. However, other silicone oils can also advantageously be used in the context of the present invention for example hexamethylcyclotrisiloxane, polydimethylsiloxane, and poly(methylphenylsiloxane).

Mixtures of cyclomethicone and isotridecyl isononanoate, and of cyclomethicone and 2-ethylhexyl isostearate, are furthermore particularly advantageous.

The oil component is furthermore advantageously selected from the group consisting of the phospholipids. The phospholipids are phosphoric esters of acylated glycerols. Of most importance among the phosphatidylcholines are, for example, lecithins, which are distinguished by the general structure

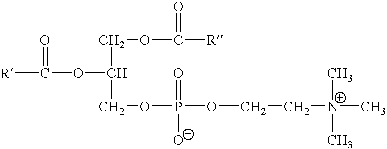

in which R′ and R″ are typically straight-chain aliphatic radicals having 15 or 17 carbon atoms and up to 4 cis-double bonds.

The combination with solubilizers, in particular in the form of the hydrogenated fatty acid monoglycerides, fatty acid diglycerides or fatty acid triglycerides, which have been ethoxylated and have a degree of ethoxylation of from 20 to 500, is preferred in the context of the invention for the use of these oil components. In particular, the raw materials PEG-200 hydrogenated glycerol palmitate, PEG-100 hydrogenated glyceryl palmitate and PEG-40 hydrogenated castor oil have proven advantageous.

By combining polymers and the abovementioned active substances and auxiliaries, it is possible to reinforce the effect and to achieve an even better, previously unattained skin sensation.

Among such polymers having at least partly quaternized nitrogen groups (referred to below as “film formers”), those which are selected from the group consisting of the substances which bear the name “polyquaternium” according to the INCI Nomenclature (International Nomenclature Cosmetic Ingredient) are preferably suitable, for example:

Polyquaternium-2 (Chemical Abstracts No. 63451-27-4, e.g. Mirapol® A-15)

Polyquaternium-5 (copolymer of acrylamide and β-methacryloxyethyltri-methylammonium methosulfate, CAS No. 26006-22-4)

Polyquaternium-6 (homopolymer of N,N-dimethyl-N-2-propen-2-ylprop-1-eneaminium chloride, CAS No. 26062-79-3, e.g. Merquat® 100

Polyquaternium-7 N,N-dimethyl-N-2-propen-2-ylprop-1-eneaminium chloride, polymer with 2-propenamide, CAS No. 26590-05-6, e.g. Merquat® S

Polyquaternium-10 quaternary ammonium salt of hydroxyethylcellulose, CAS No. 53568-66-4, 55353-19-0, 54351-50-7, 68610-92-4, 81859-24-7, e.g. Celquat® SC-230M,

Polyquaternium-11 vinylpyrrolidone/dimethylaminoethyl methacrylate copolymer/diethyl sulfate reaction product, CAS No. 53633-54-8, e.g. Gafquat® 755N

Polyquaternium-16 vinylpyrrolidone/vinylimidazolinium methochloride copolymer, CAS No. 29297-55-0, e.g. Luviquat® HM 552

Polyquaternium-17 CAS No. 90624-75-2, e.g. Mirapol® AD-1

Polyquaternium-19 quaternized water-soluble polyvinyl alcohol

Polyquaternium-20 water-dispersible quaternized polyvinyl octadecyl ether

Polyquaternium-21 polysiloxane-polydimethyl-dimethylammonium acetate copolymer, e.g. Abil® 9905

Polyquaternium-22 dimethyldiallylammonium chloride/acrylic acid copolymer, CAS No. 53694-7-0, e.g. Merquat® 280

Polyquaternium-24 polymeric quaternary ammonium salt of hydroxyethylcellulose, reaction product with an epoxide substituted by lauryldimethylammonium, CAS No. 107987-23-5, e.g. Quatrisoft® LM-200

Polyquaternium-28 vinylpyrrolidone/methacrylamidopropyltrimethylammonium chloride copolymer, e.g. Gafquat® HS-100

Polyquaternium-29 e.g. Lexquat® CH

Polyquaternium-31 CAS No. 136505-O₂-7, e.g. Hypan® QT 100

Polyquaternium-32 N,N,N-trimethyl-2-[(2-methyl-1-oxo-2-propenyl)oxy]-ethanaminium chloride, polymer with 2-propeneamide, CAS No. 35429-19-7

Polyquaternium-37 CAS No. 26161-33-1

Preferably, the polymers polyquaternium-10 and polyquaternium-22 have proven advantageous.

The cosmetic and dermatological preparations according to the invention may contain cosmetic auxiliaries, as usually used in such preparations, e.g. preservatives, bactericides, virucides, perfumes, substances for preventing foaming, dyes, pigments, further thickeners (not covered by the definition of the thickeners according to the invention), surface-active substances, emulsifiers, softening, moistening and humectant substances, antiinflammatory substances, medicaments, fats, oils, waxes and other customary constituents of cosmetic or dermatological formulation, such as alcohols, polyols, polymers, foam stabilizers, electrolytes and organic solvents.

### FORMULATION EXAMPLES

FORMULATION EXAMPLES

| | 1 | 2 | 3 | 4 |
|---|---|---|---|---|
| Sodium cocoyl glutamate | 2.5 | — | — | — |
| Sodium lauryl ether sulfate | — | 3.5 | — | — |
| Sodium lauryl sarcosinate | — | — | 5 | — |
| Sodium myristyl ether sulfate | — | — | — | 4.5 |
| Decylglycoside | 3 | 4 | — | — |
| Laurylglycoside | — | — | 3 | 3 |
| Polyquaternium-10 | 0.1 | — | — | 0.1 |
| Guar hydroxypropyltrimonium chloride | — | 0.15 | — | — |
| Polyquaternium-22 | — | — | 0.2 | — |
| PEG-200 hydrogenated glyceryl | 0.5 | — | — | — |
| palmitate | | | | |
| PEG-40 hydrogenated castor oil | 0.1 | 0.1 | 0.1 | 0.1 |
| PEG-100 hydrogenated glyceryl | — | 0.5 | — | 0.5 |
| palmitate | | | | |
| Sodium benzoate | 0.5 | 0.5 | — | 0.5 |
| Sodium salicylate | — | 0.2 | — | 0.2 |
| Methyldibromoglutaronitrile | — | — | 0.04 | — |
| Phenoxyethanol | — | — | 0.16 | — |
| Jojoba oil (Buxus Chinensis) | 0.1 | — | — | — |
| Citric acid | q.s. | q.s. | q.s. | q.s. |
| Perfume | q.s. | q.s. | q.s. | q.s. |
| Water | to | to | to | to |
| | 100 | 100 | 100 | 100 |

That which is claimed:

1. A cosmetic cleaning product, comprising:
a foamable, aqueous cleaning preparation comprising one or more nonionic surfactants and one or more anionic surfactants, and
a pumpfoamer comprising a storage container and a pump mechanism in the form of a closure and having a riser tube for foaming the cleaning preparation present in the storage container.

2. The cosmetic cleaning product as claimed in claim 1, wherein the cleaning preparation has a surfactant concentration from 2 to 15% by weight, based on the total weight of the preparation.

3. The cosmetic cleaning product as claimed in claim 1, wherein the cleaning preparation has a surfactant concentration from 4 to 9% by weight, based on the total weight of the preparation.

4. The cosmetic cleaning product as claimed in claim 1, wherein the cleaning preparation has a ratio of anionic surfactant to nonionic surfactant equal to a to b—with a and b each being from 2 to 5.

5. The cosmetic cleaning product as claimed in claim 1, wherein the cleaning preparation has a ratio of anionic surfactant to nonionic surfactant (a to b) from 5:3 to 3.5:4.

6. The cosmetic cleaning product as claimed in claim 1, wherein the cleaning preparation has a ratio of anionic surfactant to nonionic surfactant (a to b) of 2.5:3.

7. The cosmetic cleaning product as claimed in claim 1, wherein the one or more nonionic surfactants in the cleaning preparation include one or more alkylglycosides.

8. The cosmetic cleaning product as claimed in claim 7, wherein the one or more nonionic surfactants in the cleaning preparation include one or more of laurylglycoside and decylglycoside.

9. The cosmetic cleaning product as claimed in claim 1, wherein the one or more anionic surfactants in the cleaning preparation include one or more acyl glutamates.

10. The cosmetic cleaning product as claimed in claim 1, wherein the one or more anionic surfactants in the cleaning preparation include sodium cocoyl glutamate.

11. The cosmetic cleaning product as claimed in claim 1, wherein the cleaning preparation further comprises one or more of emollients, oil components, dyes, pigments, preservatives and perfumes.

12. The cosmetic cleaning product as claimed in claim 1, wherein the cleaning preparation further comprises one or more compounds selected from the group consisting of hydrogenated fatty acid monoglycerides, fatty acid diglycerides and fatty acid triglycerides, said compounds having been ethoxylated and having a degree of ethoxylation of from 20 to 500.

13. The cosmetic cleaning product as claimed in claim 1, wherein the pumpfoamer is protected from water splashes.

14. A method for cleansing the skin or hair, comprising applying to the skin or hair through the use of a pumpfoamer, a foamable, aqueous cleaning preparation comprising one or more nonionic surfactants and one or more anionic surfactants, wherein said pumpfoamer comprises a storage container and a pump mechanism in the form of a closure and having a riser tube for foaming the cleaning preparation present in the storage container.
